(19) United States
(12) Patent Application Publication
(10) Pub. No.: US 2010/0063434 A1
(43) Pub. Date: Mar. 11, 2010

(54) Foam with Antimicrobial and Hemostatic Agents

(76) Inventor: Vinesh Naik, Plains, PA (US)

Correspondence Address: AKERMAN SENTERFITT, P.O. BOX 3188, WEST PALM BEACH, FL, 33402-3188, US

(21) Appl. No.: 12/554,727

(22) Filed: Sep. 4, 2009

Related U.S. Application Data
(60) Provisional application No. 61/096,003, filed on Sep. 11, 2008.

(51) Int. Cl. A61L 15/44; A61F 13/00

(52) U.S. Cl. 602/46; 602/48

(57) ABSTRACT

An antibacterial bandage formed from at least one foam with at least one antimicrobial agent and at least one hemostatic agent is disclosed. The at least one antimicrobial agent and the at least one hemostatic agent may be mixed with materials forming the foam during manufacture of the at least one foam.

### CROSS-REFERENCE TO RELATED APPLICATION

This patent application claims the benefit of U.S. Provisional Patent Application No. 61/096,003, filed Sep. 11, 2008, which is incorporated by reference in its entirety.

### FIELD OF THE INVENTION

This invention is directed generally to medical bandages, and more particularly to medical bandages having antimicrobial properties.

### BACKGROUND

Patient health care typically involves attending to the immediate injury or injury. Bandages of a variety of configurations have been instrumental in attending to injuries in the field, for use during surgery and in other applications as well. The bandages assist in controlling bleeding and protect the wound site or surgery incision from exposure to foreign materials. Bandages also prevent medications, such as ointments, that are spread on a wound or incision from being inadvertently wiped off. Through the physical barrier created by bandages, the bandages have supported improved wound care in patients.

While conventional bandages form a physical barrier to foreign matter, wounds often remain susceptible to infection. Infections remain susceptible to pathogens until the wound heals. Thus, a need exists for an improved method of promoting an increased rate of healing together with a reduced likelihood of infection.

### SUMMARY OF THE INVENTION

This invention is directed to a foam bandage having antimicrobial and hemostatic properties for reducing the risk of infections and for fostering a more favorable healing environment. The components having the antimicrobial and hemostatic properties may be incorporated into the foam bandage during formation of the foam, thereby mixing the components through the foam material. The foam forming the bandage may be formed in any appropriate configuration, such as in a sheet or other appropriate shape and may be attached to any appropriate material to form a bandage. For instance and not by way of limitation, the foam may be attached to a self-adhering bandage, such as a bandage that includes an adhesive, or may be attached to an elastic backing material. The foam bandage may be formed in any appropriate size and may be used in any appropriate manner for wound care.

The antibacterial bandage may be formed from at least one foam formed with one or more antimicrobial agents and one or more hemostatic agents, wherein the antimicrobial agent and the hemostatic agent are mixed with materials forming the foam during manufacture of the at least one foam. The antimicrobial agent and the hemostatic agent may be mixed with materials forming the foam during manufacture of the at least one foam during the organic phase.

The foam may be hydrophobic or hydrophilic. The foam may be formed from materials selected from the group consisting of polyurethane and combinations of polyurethane, polyester and polyether. The antimicrobial agent may be formed from materials such as, but not limited to, silver, copper and zinc. The hemostatic agent may be formed from materials such as, but not limited to, chitosan, zeolite, traumadex, silica, and celite.

In some embodiments, the antimicrobial agent and the at least one hemostatic agent combined into a single component. For instance, the antimicrobial agent and the at least one hemostatic agent may be formed from silver coated, glass particles or silver impregnated, glass particles, or both. The silver coated, glass particles or silver impregnated, glass particles form between about 10 percent and about 30 percent of the foam.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a depiction of a hydrophilic foam with antimicrobial, hemostatic agents as viewed through a 15 power lens.

FIG. 2 a depiction of a hydrophilic foam with antimicrobial, hemostatic agents as viewed through a 90 power lens.

FIG. 3 is a depiction of test material for hemostatic properties of different agents.

FIG. 4 is a perspective view of an exemplary foam bandage of this invention.

### DETAILED DESCRIPTION OF THE INVENTION

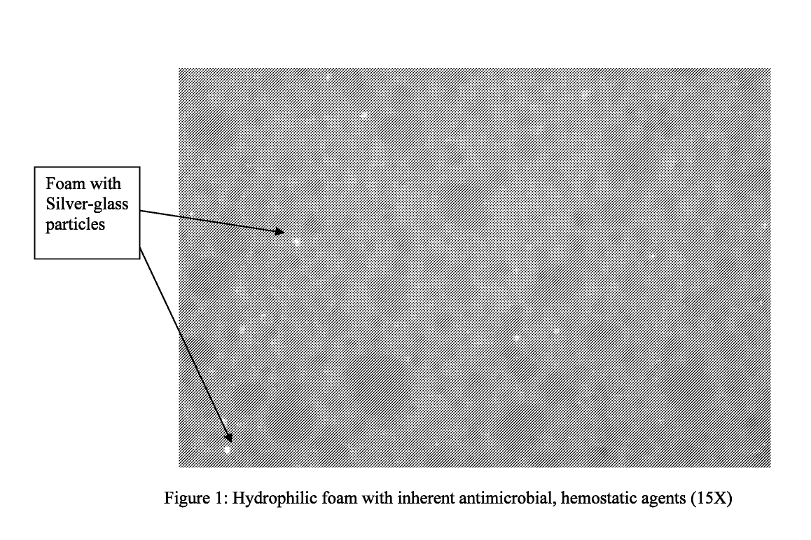

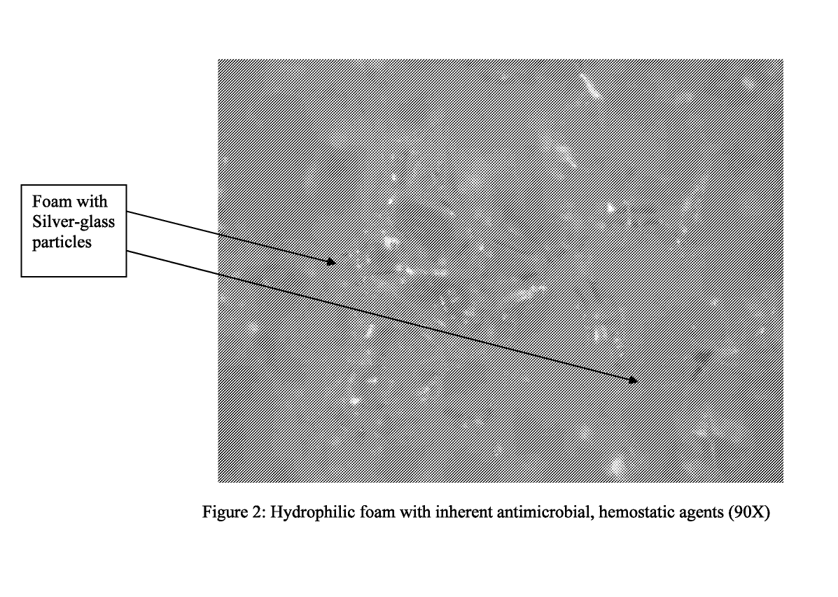

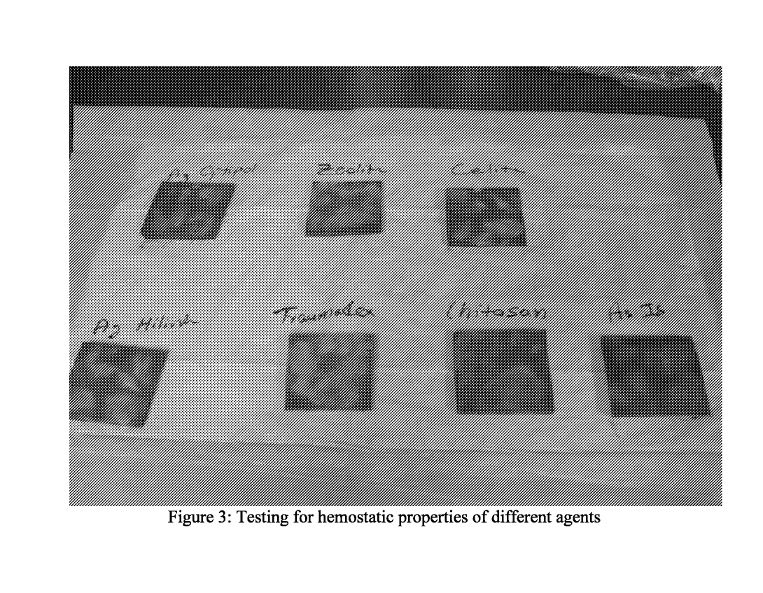

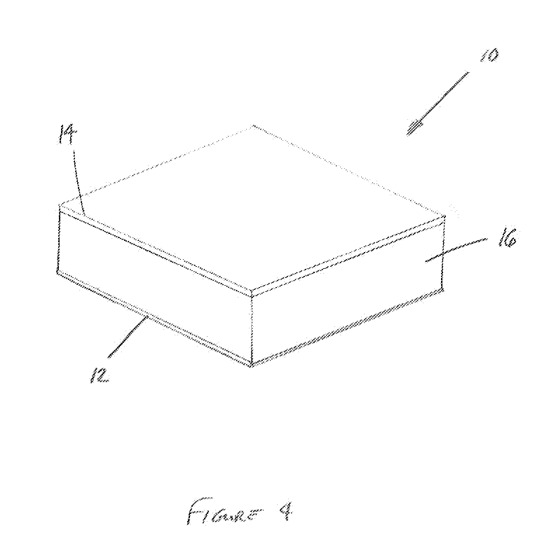

As shown in FIGS. 1-4, this invention is directed to a foam bandage 10 having antimicrobial and hemostatic properties for reducing the risk of infections and for fostering a more favorable healing environment. The foam bandage 10 may be use in a variety of applications, including, but not limited to, surgery, wound care and trauma. The components having the antimicrobial and hemostatic properties may be incorporated into the foam bandage 10 during formation of the foam 16, thereby mixing the components through the foam material. The foam 16 forming the bandage 10 may be formed in any appropriate configuration, such as in a sheet or other appropriate shape and may be attached to any appropriate material to form a bandage. For instance and not by way of limitation, the foam 16 may be attached to a self-adhering bandage, such as a bandage that includes an adhesive layer 12, or may be attached to an elastic backing material 14. The foam bandage 10 may be formed in any appropriate size and may be used in any appropriate manner for wound care.

The foam bandage 10 may be formed from foam 16 that is hydrophilic or hydrophobic. The foam 16 may be formed from materials such as, but not limited to polyurethane, a mixture of polyurethane, polyester, polyether and other appropriate materials. While the foam 16 is being manufactured, the antimicrobial materials and the hemostatic materials may be mixed together with the materials used to form the foam 16. In particular, the materials used to form the foam 16 exist in two phases, the aqueous and organic phases. The antimicrobial and hemostatic materials may be added to the materials used to form the foam 16 while in the organic phase. The antimicrobial materials may be, but are not limited to, silver, copper or zinc particles. The antimicrobial material may be formed from one or more silver coated glass particles or silver impregnated glass particles. In at least one embodiment, the silver coated glass particles may be formed form about one half formed from a glass ball and the other half formed from an antimicrobial material, such as, but not limited to, silver. In one embodiment, the glass half of the glass particle may be generally hemispherical and the antimicrobial half may be generally hemispherical as well. The amount of glass particles that may be added to the materials forming the foam 16 may be between about 10 percent and about 30 percent of the foam 16. The hemostatic particles may be, but are not limited to, chitosan, zeolite, traumadex, silica, and celite.

During the manufacturing process, antimicrobial and hemostatic agents such as but not limited to silver particles, copper particles, zinc particles, silver-glass particles with combination of chitosan, zeolite, traumadex, silica, celite, etc. may be added to aqueous or organic phase, or both, of the materials used to form the foam 16. Two phases of the material may be mixed, and the resultant solution may be poured into a tray such that a reaction may occur. Once the reaction is complete, the foam 16 has been formed made. The foam 16 may then be subjected to heat, such as, but not limited to, microwaves or a dryer, for a few minutes to evaporate any excess moisture. The foam 16 has inherent antimicrobial and hemostatic properties. The density or thickness of the foam 16 does not hinder the intrinsic properties of the foam 16 with the antibacterial and hemostatic materials.

In embodiments in which the foam 16 is a hydrophilic, the hydrophilic nature of the material forming the foam 16 may be advantageous. A test for absorption properties was conducted, and the results documented below. The results have been compared to commercially available gauze.

TABLE 1
2 × 2 Medical Grade Foam
2 × 2 Standard Gauze Dressing

| | Test | | | |
|---|---|---|---|---|
| Reagent | Amount | Residual | Adsorbed | Time |
| Silica gel | 15 ml | 10 ml | 5 ml | 1:45 |
| 5% | | | | |
| Traumadex | 15 ml | 10 ml | 5 ml | 1:15 |
| Ag | 15 ml | 3 ml | 12 ml | 0:45 |
| Highlink | | | | |
| Chitosan | 15 ml | 6 ml | 9 ml | 1:30 |
| Zeolite | 15 ml | 6 ml | 9 ml | 1:30 |
| Gauze | 15 ml | 14 ml | 1 ml | 0:15 |
| Ag | 15 ml | 6 ml | 9 ml | 1:15 |
| Opthipol | | | | |
| Celite | 15 ml | 10 ml | 5 ml | 1:15 |

I claim:

1. An antibacterial bandage, comprising:
at least one foam formed with at least one antimicrobial agent and at least one hemostatic agent, wherein the at least one antimicrobial agent and the at least one hemostatic agent are mixed with materials forming the foam during manufacture of the at least one foam.

2. The antibacterial bandage of claim 1, wherein the at least one foam is hydrophobic.

3. The antibacterial bandage of claim 1, wherein the at least one foam is hydrophilic.

4. The antibacterial bandage of claim 1, wherein the at least one foam is formed from materials selected from the group consisting of polyurethane and combinations of polyurethane, polyester and polyether.

5. The antibacterial bandage of claim 1, wherein the at least one antimicrobial agent is formed from materials selected from the group consisting of silver, copper and zinc.

6. The antibacterial bandage of claim 1, wherein the at least one hemostatic agent is selected from at least one of the group consisting of chitosan, zeolite, traumadex, silica, and celite.

7. The antibacterial bandage of claim 1, wherein the at least one antimicrobial agent and the at least one hemostatic agent is formed from silver coated, glass particles.

8. The antibacterial bandage of claim 7, wherein the silver coated, glass particles form between about 10 percent and about 30 percent of the at least one foam.

9. The antibacterial bandage of claim 1, wherein the at least one antimicrobial agent and the at least one hemostatic agent is formed from silver impregnated, glass particles.

10. The antibacterial bandage of claim 9, wherein the silver impregnated, glass particles form between about 10 percent and about 30 percent of the at least one foam.

11. The antibacterial bandage of claim 1, wherein the at least one antimicrobial agent and the at least one hemostatic agent is mixed with materials forming the foam during manufacture of the at least one foam during the organic phase.

12. An antibacterial bandage, comprising:
at least one foam material;
at least one formed with at least one antimicrobial agent and at least one hemostatic agent, wherein the at least one antimicrobial agent and the at least one hemostatic agent are mixed with materials forming the foam during the organic phase during manufacture of the at least one foam;
wherein the at least one antimicrobial agent and the at least one hemostatic agent is formed from silver coated, glass particles.

13. The antibacterial bandage of claim 12, wherein the at least one foam is hydrophobic.

14. The antibacterial bandage of claim 12, wherein the at least one foam is hydrophilic.

15. The antibacterial bandage of claim 12, wherein the at least one foam is formed from materials selected from the group consisting of polyurethane and combinations of polyurethane, polyester and polyether.

16. The antibacterial bandage of claim 12, wherein the silver coated, glass particles form between about 10 percent and about 30 percent of the at least one foam.

17. An antibacterial bandage, comprising:
at least one foam material;
at least one formed with at least one antimicrobial agent and at least one hemostatic agent, wherein the at least one antimicrobial agent and the at least one hemostatic agent are mixed with materials forming the foam during the organic phase during manufacture of the at least one foam;
wherein the at least one antimicrobial agent and the at least one hemostatic agent is formed from silver impregnated, glass particles.

18. The antibacterial bandage of claim 17, wherein the at least one foam is hydrophilic.

19. The antibacterial bandage of claim 17, wherein the at least one foam is formed from materials selected from the group consisting of polyurethane and combinations of polyurethane, polyester and polyether.

20. The antibacterial bandage of claim 17, wherein the silver coated, glass particles form between about 10 percent and about 30 percent of the at least one foam.
